(19) United States
(12) Patent Application Publication
(10) Pub. No.: US 2006/0275364 A1
(43) Pub. Date: Dec. 7, 2006

(54) Flexible Solid Dosage Forms and Methods of Making and Using the Same

(76) Inventors: Salah U. Ahmed, New City, NY (US); Lianli Li, Pomona, NY (US); Tahseen A. Chowdhury, Washington Township, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/420,423

(22) Filed: May 25, 2006

Related U.S. Application Data
(60) Provisional application No. 60/684,523, filed on May 26, 2005.

(51) Int. Cl. A61K 9/20; A61K 36/47

(52) U.S. Cl. 424/464; 424/731

(57) ABSTRACT

The present invention provides flexible solid dosage forms that include an active agent, gelatin, and a polyol or saccharide. The present invention also provides methods of making flexible solid dosage forms.

### CROSS REFERENCE TO RELATED APPLICATIONS

This application claims the benefit of the filing date of U.S. application No. 60/684,523, filed May 26, 2005, the contents of which are hereby incorporated by reference in its entirety.

### BACKGROUND OF THE INVENTION

1. Field of the Invention

The present invention relates to flexible solid dosage forms and methods of making and using the same.

2. Background Art

Solid dosage forms designed for oral delivery of an active agent can be buccal tablets, sublingual tablets, orally disintegrating or soluble tablets, chewable tablets, lozenges, or lollipops, and oral strips or films.

Some oral delivery formulations are developed to provide sustained release of the active agent while others provide quick disintegration or dissolution in the oral cavity.

Some oral delivery formulations are designed to be placed on the buccal mucosa or under the tongue where they dissolve slowly or quickly, allowing the active agent to be absorbed through the lining of the mouth. Other oral delivery formulations disintegrate or dissolve quickly on the tongue upon contact with the saliva in the mouth, and then swallowed, allowing the active agent to be absorbed from the gastrointestinal tract. Effervescence has been used to enhance the quick disintegration of the formulation. Some oral delivery formulations dissolve quickly on the tongue and leave freshness in the mouth.

Oral films currently available in the market such as Listerine PocketPaks® Oral Strips (Pfizer) are typically manufactured by a film/patch preparation process involving coating a polymer/solvent dispersion onto a linear base and then cutting the resulting film into the appropriate size to obtain the final oral film strips.

Kosmos Pharma has developed stamp-sized film strips, called FDTAB, that are thin and flexible and can dissolve in the mouth without the need for water or chewing and can contain a drug.

### BRIEF SUMMARY OF THE INVENTION

The present invention is directed to a solid dosage form comprising a substantially homogeneous mixture of an active agent, gelatin, and a polyol or saccharide, wherein the solid dosage form is not rigid, but is flexible. In some embodiments, the solid dosage form can be erosion controlled and can erode at a rate of about 0.5 mg/min/cm² to about 10 mg/min/cm².

In some embodiments, the active agent in the solid dosage form can be selected from the group consisting of a drug, vitamin, mineral, dietary supplement, and combinations thereof. In some embodiments, the active agent is a drug.

In some embodiments, the polyol in the solid dosage form can be selected from the group consisting of sorbitol, mannitol, xylitol, maltitol, lactitol, isomalt, PEG (polyethylene glycol), and combinations thereof. In some solid dosage forms, the polyol is sorbitol.

The solid dosage form can further comprise a sweetener. The sweetener can be selected from the group consisting of aspartame, sucralose, sucrose, saccharin, sodium saccharin, and combinations thereof. In some solid dosage forms, the sweetener is aspartame.

The solid dosage form can further comprise a flavoring agent. The flavoring agent can be selected from the group consisting of orange flavor, lemon flavor, strawberry flavor, peppermint flavor, mint flavor, and combinations thereof. In some solid dosage forms, the flavoring agent is orange flavor.

The solid dosage form can further comprise a lubricant. The lubricant can be selected from the group consisting of stearic acid, colloidal silicon dioxide, hydrogenated vegetable oil, silica, magnesium stearate, and combinations thereof. In some solid dosage forms, the lubricant is stearic acid, colloidal silicon dioxide, or a combination thereof.

The solid dosage form can further comprise a polymer selected from the group consisting of sodium alginate, carbomer, microcrystalline cellulose, polyvinyl pyrrolidone, polyethylene oxide (PEO), glycerin, hydroxypropyl methylcellulose (HPMC), hydroxypropyl cellulose (HPC), hydroxyethyl cellulose (HEC), polyvinyl alcohol (PVA), sodium carboxymethylcellulose (Na CMC), tragacanth, and combinations thereof.

The solid dosage form can also further comprise an excipient selected from the group consisting of triacetin, triethyl citrate, dibutyl sebacate, castor oil, PEG 400, propylene glycol, and combinations thereof.

The solid dosage forms of the present invention can be used for various delivery routes. In some embodiments, the solid dosage forms are oral tablets. In some embodiments, the solid dosage forms are vaginal rings or suppositories.

In some embodiments, the oral tablet can be about 0.1 mm to about 5 mm in thickness. In some embodiments, the vaginal ring can be about 0.1 to about 0.5 inches in thickness. In some embodiments, the solid dosage form can have an elongation of about 10% to about 150%.

In some embodiments, the solid dosage forms can be further coated with typical coating agents such as hydroxypropyl methylcellulose (HPMC), hydroxypropyl cellulose (HPC), and other coating agents known in the art.

The present invention is also directed to a method of making a flexible solid dosage form, the method comprising: mixing an active agent, gelatin, and a polyol or saccharide to form a substantially homogenous mixture; compressing the substantially homogeneous mixture into a solid dosage form; and curing the solid dosage form.

The present invention is also directed to a method of making a flexible solid dosage form, the method comprising (a) dissolving an active agent in a solvent to form a solution; (b) mixing the solution with a polyol or saccharide to form granules; (c) drying and mixing the granules with gelatin; (d) compressing the mixture into a solid dosage form; and (e) curing the solid dosage form.

The invention is also directed to a method of making a flexible solid dosage form, the method comprising (a) mixing a gelatin, and a polyol or saccharide to form a substantially homogenous mixture; (b) compressing the substantially homogeneous mixture into a solid dosage form; (c) curing the solid dosage form; and (d) soaking the cured solid dosage form in a solution comprising an active agent.

The method can further comprise mixing a sweetener, a flavoring agent, a lubricant, or a combination thereof into the mixture prior to compressing the mixture.

The method can also further comprise mixing a polymer selected from the group consisting of sodium alginate, carbomer, microcrystalline cellulose, polyvinyl pyrrolidone, polyethylene oxide (PEO), glycerin, hydroxypropyl methylcellulose (HPMC), hydroxypropyl cellulose (HPC), hydroxyethyl cellulose (HEC), polyvinyl alcohol (PVA), sodium carboxymethylcellulose (Na CMC), tragacanth and combinations thereof, into the mixture prior to compressing the mixture. An excipient selected from the group consisting of triacetin, triethyl citrate, dibutyl sebacate, castor oil, PEG 400, propylene glycol, and combinations thereof, can also be mixed into the mixture prior to compressing the mixture.

In some embodiments, the mixing can be carried out using a high shear mixer. In some embodiments, the curing can be carried out at about 30° C. to about 60° C. In some embodiments, the curing can be carried out at about 65% to about 85% relative humidity.

The method can further comprise drying the solid dosage form. The drying can be carried out at about 30° C. to about 70° C. In some embodiments, the drying can be carried out for a period of about 30 minutes to about 6 hours.

The method can further comprise cooling the solid dosage form. In some embodiments, the cooling can be carried out at room temperature. In some embodiments, the cooling can be for about 30 minutes to about 2 hours.

The present invention is also directed to a solid dosage form comprising a substantially homogeneous mixture of an active agent, gelatin, a polyol or saccharide, and a preservative, wherein the solid dosage form is flexible. In some embodiments, the preservative can be an antimicrobial agent, an antioxidant, a chelating agent, or a combination thereof. In some embodiments, the antimicrobial agent is selected from the group consisting of benzalkonium chloride, cetalokonium chloride, benzoates, benzyl alcohol, methyl paraben, propyl paraben, alkaly gallates, hydroxybenzoates and salts thereof, phenyl mercuric salts, sodium hypocholorite, and acetic acid. In further embodiments, the antimicrobial agent is selected from the group consisting of benzoates, benzyl alcohol, methyl paraben, propyl paraben, alkaly gallates, and hydroxybenzoates and salts thereof. In some embodiments, the antioxidant is selected from the group consisting of alkaly gallates, ascorbyl palmitate, butylated hydroxy anisole, butylated hydroxy toluene, sodium bi sulphite, sodium meta bi sulphite, and potassium meta bi sulphite. In some embodiments, the chelating agent is ethylenediaminetetraacetic acid (EDTA).

### BRIEF DESCRIPTION OF THE DRAWINGS/FIGURES

FIG. 1 is a flow chart illustrating a method of making a flexible tablet using the compression method in accordance with the present invention.

FIG. 2 is a flowchart illustrating a method of making a flexible tablet using a granulation compression method in accordance with the present invention.

FIG. 3 is a flowchart illustrating a method of making a flexible tablet using the soaking method in accordance with the present invention.

FIG. 4 is an electron microscope image of gelatin 75 Bloom granules prior to mixing with sorbitol.

FIG. 5 is an electron microscope image of sorbitol instant granules prior to mixing with gelatin.

FIG. 6 is an electron microscope image of a cross-section of a tablet formed by mixing gelatin and sorbitol and compressing (but prior to curing the tablet).

FIG. 7 is an electron microscope image of a cross-section of a tablet formed by mixing gelatin and sorbitol, compressing, and curing the tablet.

FIG. 8 shows strain-time curves of flexible oral tablets after curing.

FIG. 9 shows the erosion of a flexible oral tablet with time.

FIG. 10 shows a stress-strain curve of flexible oral tablets after curing.

FIG. 11 shows the percentage of dissolution of olanzapine from two different formulations of flexible oral tablets as a function of time.

FIG. 12 shows a heat flow-temperature curve obtained from Differential Scanning Calorimetry using, as samples, 1) sorbitol, 2) gelatin, 3) a dry mix of sorbitol and gelatin in a 1:1 ratio by weight that has been compressed into a rigid tablet, before curing, and 4) a flexible tablet containing sorbitol and gelatin in a 1:1 ratio by weight, after curing.

FIG. 13 shows an X-ray diffraction graph of 1) a dry mix of sorbitol and gelatin in a 1:1 ratio by weight that has been compressed into a rigid tablet, before curing, and 2) a flexible tablet containing sorbitol and gelatin in a 1:1 ratio by weight, after curing.

FIG. 14 shows the dissolution profile of a flexible oral tablet containing estradiol made by (a) the granulation compression method (denoted by solid squares), and (b) the soaking method (denoted by solid diamonds) as described in Example 11.

### DETAILED DESCRIPTION OF THE INVENTION

The present invention is directed to flexible solid dosage forms and methods of making and using such solid dosage forms. The flexible solid dosage forms of the present invention are soluble and erodible. The flexible solid dosage forms can be used for multiple purposes, such as for oral transmucosal drug delivery, oral care, oral freshener, vaginal drug delivery, vaginal care, and rectal drug delivery. The flexible solid dosage forms of the present invention can be used, for example, in the administration of an active agent via the buccal cavity. Upon exposure to saliva, the flexible solid dosage form of the present invention hydrates, and adheres to the tongue or oral mucosa. The solid dosage forms of the present invention can also be used in the vaginal or rectal administration of an active agent, via a flexible vaginal ring or suppository. Therefore, the present invention provides a solid dosage form comprising a substantially homogeneous mixture of an active agent, gelatin, and a polyol or saccharide, wherein the solid dosage form is not rigid, but is flexible. The flexible solid dosage forms can be provided in the form of, for example, orally soluble tablets and chewable tablets, as well as vaginal rings and suppositories.

The solid dosage forms of the present invention can be erosion controlled. By this, the solid dosage forms will erode, either slowly or quickly (depending on the desired speed at which the active agent is release from the solid dosage form). The erosion pattern of the solid dosage forms can also depend on the thickness, size, and composition of the solid dosage forms. An eroding solid dosage form of the present invention will not substantially disintegrate. That is, the eroding solid dosage form will not substantially dislocate and break up into small pieces upon contact with an aqueous media as would a disintegrating tablet. Unlike a disintegrating tablet, the aqueous media dissolves the solid dosage forms of the present invention from the surface of the solid dosage forms without dislocating the solid dosage forms into smaller pieces.

The amount of time required for substantially complete erosion of the solid dosage forms of the present invention depends on various factors, such as, but not limited to, the weight, surface area, and composition of solid dosage forms, and the conditions of the environment in which the erosion takes place. Erosion studies of the flexible solid dosage forms of the present invention can be performed, for example, in USP dissolution apparatus II (paddle method) at about 37° C. and about 50 rpm in 900 ml water. The erosion rate can then be calculated in units such as mg/min/cm² to account for the erosion time (in minutes (min)) as a function of the weight (in milligrams (mg)) and surface area (in square centimeters (cm²)) of the solid dosage form. When the flexible solid dosage forms of the present invention are subjected to a test in USP dissolution apparatus II (paddle method) at about 37° C. and about 50 rpm in 900 ml water, the solid dosage forms can have an erosion rate of, but not limited to, about 0.5 mg/min/cm² to about 10 mg/min/cm², about 1 mg/min/cm² to about 6 mg/min/cm², or about 1 mg/min/cm² to about 4 mg/min/cm². The erosion rate can vary based on the formulation composition, such as the content of Cab-O-Sil® (Cabot Corporation, Boston, Mass.), microcrystalline cellulose, and other excipients in the solid dosage forms of the present invention.

As used herein, “about” refers to plus or minus 10% of the indicated number. For example, “about 10 mg/min/cm²” indicates a range of 9 mg/min/cm² to 11 mg/min/cm².

The active agent in the solid dosage forms of the present invention can be any active agent suitable for administration via the dosage forms of the present invention. For example, the active agent can be, but is not limited to, a drug, vitamin, mineral, dietary supplement, or combinations thereof. In some embodiments, the active agent is a drug such as fentanyl citrate. The active ingredient can also be, but is not limited to, systematically distributable pharmaceutical ingredients, vitamins, minerals and dietary supplements, as well as non-systematically distributable pharmaceutical ingredients. Pharmaceutical ingredients include gastrointestinal function conditioning agents, antacids, analgesics, anti-inflammatories, antipsychotics, antipyretics antibiotics, antimicrobials, laxatives, anorexics, antihistamines, antiasthmatics, antidiuretics, antiflatuents, antimigraine agents, antispasmodics, sedatives, antihyperactives, antihypertensives, tranquilizers, decongestants, beta blockers and combinations thereof. Representative examples include gastrointestinal function conditioning agents such as bromopride, metoclopramide, cisapride and domperidone; anti-inflammatory agents such as aceclofenac, diclofenac, flubiprofen, sulindac and celecoxib; analgesics acetaminophen and aspirin; agents for erectile dysfunction therapy, sildenafil and apomorphine; the anti-migraines, sumatriptan and ergotamin; anti-cholinergic agents, scopolamine hydrobromide; the antihistaminic agents, loratadine, fexofenadine and cetirizine; the cardiovascular agents, nitroglycerine and isosorbide dinitrate; the diuretics, furocemide and spironolactone; the anti-hypertensive agents, nimodipine, propranolol, amlodipine, felodipine, nifedipine, captoprile, ramiprile, atenolol and diltiazem; the anti-hypolipidemic agents, lovastatin, simvastatin, atrovastatin and pravastatin; the anti-ulcer agents, cimetidine, ranitidine, famotidine, omeprazole and lansoprazol; the anti-emetics, meclizine hydrochloride, ondansetron, granisetron, ramosetron and tropisetron; the anti-asthmatic agents, aminophylline, theophylline, terbutaline, fenoterol, formoterol and ketotifen; the anti-psychotics, clonazepam, olanzapine and risperidone; the anti-depressants, mirtazapine, fluoxetine and sertraline; the vitamins, Vit B1, B2, B6, B12 and C; the anti-thrombotic agents, sulfinpyrazone, dipyridamole and ticlopidine; the chemotherapeutic agents, cefaclor, bacampicillin, sulfamethoxazole and rifampicin; the hormones, dexamethasone and methyltestosterone; the anthelmintic agents, piperazine, ivermectine and mebendazole; and the anti-diabetic agents, acarbose, gliclazid and glipizid; drugs useful for Alzheimer's, memantin, donepezil, galantamine, galantamine hydrobromide, rivastigmine; drugs useful for Parkinson's disease, pramipexole; drugs useful for pain management, alprazolam, tamsolosin, alfuzosin, fentanyl; hormones, cyproterone acetate, oxadralone; antihypertensives, clonidine; psychostimulants, modafinil; drugs for relief of heartburn, lanzoprazole. A drug product that has a high first pass effect is also suitable as an active agent for the formulations of the present invention. For example, testosterone and estradiol are also suitable active agents for the formulations of the present invention.

The amount of active agent present in the solid dosage forms can be, but is not limited to, about 0.1% to about 20% by weight of the solid dosage form, about 1% to about 10% by weight of the solid dosage form, about 3% to about 7% by weight of the solid dosage form, or about 5% by weight of the solid dosage form.

The solid dosage forms of the present invention further includes gelatin. Gelatin is a protein product derived from the partial hydrolysis of collagen from skin, bones, cartilage, etc. of animals. Gelatin can be created by boiling animal connective tissue. Commercially, gelatin is available in many different types and grades and are used in many food and non-food products. In the pharmaceutical field, gelatin has been used in various manners, such as a carrier or a coating material. One of the most important aspects of gelatin is its gel strength. When the gel strength is determined by the standard method (see e.g., “Methods for sampling and testing gelatine,” BS 757, Gr. British Standards Institution, 1975), the gel strength is called Bloom Strength. This is the force in grams required to press a 12.5 mm diameter plunger 4 mm into 112 g of a standard 6⅔% w/v gelatin gel at 10° C.

The gelatin in the solid dosage forms of the present invention acts as a low melting point polymer that forms an irreversible, flexible mass with a polyol or saccharide under heat and/or moisture. Examples of suitable gelatin that can be used in the solid dosage forms of the present invention include, but are not limited to, 50 to 275 Bloom gelatin, such as 60 Bloom, 70 Bloom, 75 Bloom, 150 Bloom, 175 Bloom, and 275 Bloom gelatin. The amount of gelatin present in the solid dosage forms of the present invention can be, but is not limited to, about 20% to about 80% by weight of the solid dosage form, about 30% to about 60% by weight of the solid dosage form, about 35% to about 55% by weight of solid dosage form, about 40% to about 50% by weight of the solid dosage form, or about 44% to about 47% by weight of the solid dosage form.

The flexible solid dosage forms of the present invention can also contain a polyol or saccharide that loses its crystalline property and forms an irreversible, flexible mass with the gelatin under heat and/or moisture. Polyols are low molecular weight, water-soluble components with multiple hydroxyl groups. Examples of polyols include, but are not limited to, sugar alcohols (sorbitol, mannitol, xylitol, maltitol, lactitol, isomalt, and combinations thereof) and PEG (polyethylene glycol). In some embodiments, the polyol is sorbitol. Examples of saccharides include, but are not limited to, polydextrose, sucrose, tagatose, galactose, maltodextrin, and combinations thereof. Polydextrose is synthesized from dextrose (glucose) and contains about 10% sorbitol and 1% citric acid.

The amount of the polyol or saccharide present in the solid dosage forms of the present invention can be, but is not limited to, about 20% to about 80% by weight of the solid dosage form, about 30% to about 60% by weight of the solid dosage form, about 35% to about 55% by weight of the solid dosage form, about 40% to about 50% by weight of the solid dosage form, or about 44% to about 47% by weight of the solid dosage form.

The solid dosage forms of the present invention can further include a sweetener. Suitable sweeteners for use in the solid dosage forms of the present invention include, but are not limited to, aspartame, sucralose, sucrose, saccharin, sodium saccharin, and combinations thereof. In some embodiments, the sweetener is aspartame. The amount of sweetener present in the solid dosage forms of the present invention can be, but is not limited to, about 0.1% to about 10% by weight of the solid dosage form, about 1% to about 5% by weight of the solid dosage form, about 2% to about 4% by weight of the solid dosage form, or about 3% by weight of the solid dosage form.

The solid dosage forms of the present invention can additionally include a flavoring agent. Suitable flavoring agents include, but are not limited to, orange flavor, lemon flavor, strawberry flavor, peppermint flavor, mint flavor, and combinations thereof. In some embodiments, the flavoring agent is orange flavor. The amount of flavoring agent present in the solid dosage forms of the present invention can be, but is not limited to, about 0.05% to about 5% by weight of the solid dosage form, about 0.1% to about 3% by weight of the solid dosage form, about 0.5% to about 2% by weight of the solid dosage form, or about 1% by weight of the solid dosage form.

A lubricant can also be included in the solid dosage forms of the present invention. Suitable lubricants include, but are not limited to, stearic acid, hydrogenated vegetable oil, silica, magnesium stearate, and combinations thereof. Colloidal silicon dioxide such as Cab-O-Sil® (Cabot Corporation, Boston, Mass.), for example, can also be used as a lubricant in the solid dosage forms. The amount of lubricant present in the solid dosage forms of the present invention can be, but is not limited to, about 0.1% to about 10% by weight of the solid dosage form, about 1% to about 8% by weight of the solid dosage form, about 2% to about 6% by weight of the solid dosage form, or about 3% to about 5% by weight of the solid dosage form.

Other polymers such as sodium alginate, carbomers such as Carbopol® (Noveon, Cleveland, Ohio), microcrystalline cellulose, polyvinyl pyrrolidone, polyethylene oxide (PEO), glycerin, hydroxypropyl methylcellulose (HPMC), hydroxypropyl cellulose (HPC), hydroxyethyl cellulose (PEC), polyvinyl alcohol (PVA), sodium carboxymethylcellulose (Na CMC), tragacanth, and combinations thereof, can be included in the solid dosage forms of the present invention. Other excipients such as Triacetin, Triethyl citrate, Dibutyl sebacate, Castor oil, PEG 400, Propylene glycol, and combinations thereof, can also be included in the solid dosage forms of the present invention.

The solid dosage forms of the present invention are thin and flexible. The thickness of the tablets can be, but is not limited to, about 0.1 mm to about 5 mm, about 0.5 mm to about 4 mm, about 1 mm to about 3 mm, or about 2 mm. The thickness of the vaginal rings can be, but is not limited to, about 0.10 inches to about 0.50 inches, about 0.15 inches to about 0.40 inches, about 0.20 inches to about 0.30 inches, about 0.25 inches to about 0.28 inches, or about 0.27 inches.

The solid dosage form thickness, surface area, diameter, volume and density can be adjusted in relation to one another. For example, thicker solid dosage forms or a solid dosage form with greater surface area (greater diameter) can accommodate greater solid dosage form weight, assuming the density of the solid dosage form stays constant. An equation that can be used for solid dosage form weight calculation is: W=ρ(πr²)h=ρ(πd²/4)h, where W is the solid dosage form weight, ρ is the density of the solid dosage form, r is the radius of the solid dosage form, and h is the thickness of the solid dosage form.

The solid dosage forms according to the present invention are not rigid and exhibit a great degree of flexibility. Flexibility refers to the ability of a solid to withstand stress and strain without being damaged or broken. Stress is the force applied per unit area of a cross-section that causes deformation. The effect of stress is deformation or strain. Strain is the elongation or increase in the length in the solid relative to its original length. Thus, a measurement of the percentage of elongation that a solid is capable of prior to breaking is indicative of the flexibility of the solid. The greater the percentage of elongation of a solid, the more flexible the solid is. The measurement of mechanical properties in a solid is disclosed, for example, in “Polymer Science”, Chapter 20, Physical Pharmacy, 4^{th} ed., Martin, Alfred, et al., eds., pp. 575-578 (1993).

The % elongation (% strain) of the flexible solid dosage forms can be examined by using, e.g., a Dynamic Mechanical Analyzer (DMA) Q 800 (New Castle, Del.). The strain-time curves and % elongation can be obtained under simple low stress and displacement time. The effect of stress is deformation or strain. Strain in tension is called elongation. Elongation (%)=[(L−L₀)/L₀]×100, in which L is the length under a given tensile stress and L₀ is the original length of the flexible solid dosage form.

In some embodiments, the percentage of elongation of a flexible solid dosage form according to the present invention under a stress of about 1 millipascal (mPa) for about 1 minute can be, but is not limited to, about 10% to about 150%, about 50% to about 140%, about 70% to about 130%, about 90% to about 125%, about 100% to about 120%, about 105% to about 118%, or about 110% to about 116%. A similar solid dosage form that has not been cured can typically have an elongation of less than 1% (e.g., 0.6%) under the same amount of stress for the same amount of time. The percentage of elongation of a flexible solid dosage form according to the present invention under a stress of about 0.5 mPa for about 5 minutes can be, but is not limited to, about 10% to about 120%, about 50% to about 110%, about 70% to about 105%, about 80% to about 100%, about 90% to about 99%, or about 95% to about 98%. The percentage of elongation of a flexible solid dosage form according to the present invention under a stress of about 0.5 mPa for about 2 minutes can be, but is not limited to, about 10% to about 100%, about 30% to about 90%, about 50% to about 85%, about 70% to about 80%, or about 75% to about 77%. The percentage of elongation of a flexible solid dosage form according to the present invention under a stress of about 0.75 mPa for about 1 minute can be, but is not limited to, about 10% to about 100%, about 30% to about 90%, about 50% to about 85%, about 70% to about 80%, or about 75% to about 77%. The percentage of elongation of a flexible solid dosage form according to the present invention under a stress of about 0.5 mPa for about 1 minute can be, but is not limited to, about 10% to about 100%, about 30% to about 90%, about 50% to about 80%, about 60% to about 70%, or about 63% to about 65%.

The flexibility of the solid dosage forms can depend on the amount of polyol or saccharide present. Generally, greater amounts of polyol or saccharide present in the solid dosage forms contribute to greater flexibility of the solid dosage forms. However, a suitable balance between the amount of polyol or saccharide and gelatin present in the dosage forms can be achieved for the desired amount of flexibility. The ratio of polyol or saccharide to gelatin by weight in the solid dosage forms of the present invention can be about 10:1 to 1:10, about 5:1 to about 1:5, about 3:1 to about 1:3, about 2:1 to about 1:2, or about 1:1.

The solid dosage forms according to the present invention exhibit rubber-like characteristics. Some characteristic properties of rubber include: (1) capability to undergo very high deformations, up to several fold in simple tension, (2) the highly nonlinear stress-strain plots, and (3) nearly incompressible. Characteristics of rubber are discussed, for example, in S. Burtscher, et al., “Mechanical aspects of high damping rubber,” 2nd Int. PhD Symposium in Civil Engineering (1998).

The solid dosage form of the present invention comprises a substantially homogeneous mixture of an active agent, gelatin, and a polyol or saccharide. “Homogeneous mixture” refers to a composition of two or more substances, each substance having a uniform distribution throughout the composition, e.g., the homogeneous mixture does not have layers.

In some embodiments, the solid dosage forms can be further coated with typical coating agents such as hydroxypropyl methylcellulose (HPMC), hydroxypropyl cellulose (HPC), and other coating agents known in the art.

The present invention also provides methods of making the solid dosage forms. Some aspects of the present invention use a “compression method” for making the solid dosage forms of the present invention. The compression method of making a solid dosage form includes mixing an active agent, gelatin, and a polyol or saccharide to form a substantially homogeneous mixture; compressing the substantially homogeneous mixture into a solid dosage form; and curing the solid dosage form.

The compression method of the present invention provides a cost effective means of manufacturing flexible solid dosage forms such as flexible tablets or films. Thus, for example, a flexible tablet or film for absorption of an active agent through the oral mucosal can be produced by the compression method of the present invention.

Some aspects of the present invention use a “granulation compression method” for making the solid dosage forms of the present invention. The granulation compression method of the present invention includes (a) dissolving an active agent in a solvent to form a solution; (b) mixing the solution with a polyol or saccharide to form granules; (c) drying and mixing the granules with gelatin; (d) compressing the mixture into a solid dosage form; and (e) curing the solid dosage form. The term “solution” refers to a composition formed by mixing a desired substance with a liquid or solid, the resulting composition being a substantially homogeneous single-phase composition.

Some aspects of the present invention use a “soaking method” for making the solid dosage forms of the present invention. The soaking method of present invention includes (a) mixing a gelatin, and a polyol or saccharide to form a substantially homogenous mixture; (b) compressing the substantially homogeneous mixture into a solid dosage form; (c) curing the solid dosage form; and (d) soaking the cured solid dosage form in a solution comprising an active agent. The term “soaking” refers to the placement of the solid dosage form in contact with a liquid composition resulting in the liquid composition wetting or permeating the solid dosage form.

The present invention also provides a solid dosage form made by a method of the invention as described herein, and uses thereof.

Other components, such as a sweetener, a flavoring agent, a lubricant, and combinations thereof can also be mixed into the homogeneous mixture prior to compressing the mixture using the compression method, granulation compression method, or the soaking method described herein. In some embodiments, the mixing is accomplished using a high shear mixer such as a Collette high shear mixer.

Curing provides the heat and moisture necessary for the gelatin and the polyol or saccharide to form an irreversible flexible mass. The solid dosage forms become rubber-like flexible solid dosage forms after curing with heat and moisture. The curing also ensures that the active agent, gelatin and polyol or saccharide form a substantially homogeneous mixture.

According to the methods of the present invention, the temperature at which curing is carried out can be, but is not limited to, about 30° C. to about 60° C., about 30° C. to about 50° C., about 35° C. to about 45° C., about 37° C. to about 43° C., or about 40° C. The relative humidity at which curing is carried out can be, but is not limited to, about 65% to about 85%, about 70% to about 80%, about 73% to about 77%, or about 75%. For oral tablets, the curing can be carried out for a period of, but not limited to, about 2 hour to about 10 hours, about 3 hours to about 9 hours, about 4 hours to about 8 hours, about 5 hours to about 7 hours, or about 6 hours. For vaginal rings, the curing can be carried out for a period of, but not limited to, about 10 hours to about 24 hours, about 12 hours to about 22 hours, about 14 hours to about 20 hours, about 15 hours to about 18 hours, or about 16 hours.

The curing of the solid dosage forms can be followed either by drying and/or cooling the solid dosage forms. The drying of the solid dosage forms can be carried out at a temperature of, but not limited to, about 30° C. to about 70° C., about 50° C. to about 70° C., about 55° C. to about 65° C., about 57° C. to about 63° C., or about 60° C. The drying can be carried out for a period of time of, but not limited to, about 30 minutes to about 6 hours, about 1 hour to about 5 hours, about 2 hours to about 4 hours, or about 3 hours.

The flexible solid dosage forms of the present invention can further include a preservative. A preservative can act to preserve the gelatin in the solid dosage forms. Preservatives suitable for use in the flexible solid dosage forms of the present invention include antimicrobial agents, antioxidants, and chelating agents. Examples of antimicrobial agents that can be used include, but are not limited to, benzalkonium chloride, cetalokonium chloride, benzoates (e.g. sodium benzoates), benzyl alcohol, methyl paraben, propyl paraben, alkaly gallates, hydroxybenzoates and salts thereof (e.g. methyl or propyl hydroxybenzoates and salts thereof), phenyl mercuric salts (e.g. borates or nitrates), sodium hypocholorite, and acetic acid. In some embodiments, the preservative can be, but is not limited to, benzoates (e.g. sodium benzoates), benzyl alcohol, methyl paraben, propyl paraben, alkaly gallates, and hydroxybenzoates and salts thereof (e.g. methyl or propyl hydroxybenzoates and salts thereof). Examples of antioxidants that can be used include, but are not limited to, alkaly gallates, ascorbyl palmitate, butylated hydroxy anisole, butylated hydroxy toluene, sodium bi sulphite, sodium meta bi sulphite, and potassium meta bi sulphite. An example of a chelating agent that can be used is ethylenediaminetetraacetic acid (EDTA). The amount of preservatives present in the solid dosage forms of the present invention can be, but is not limited to, about 0.01% to about 2% by weight of the solid dosage form, about 0.05% to about 1.5% by weight of the solid dosage form, or about 0.1% to about 1% by weight of the solid dosage form.

The cooling of the solid dosage forms can be carried out at room temperature, but can also be carried out at other temperatures, if desired. The amount of time the cooling is carried out can be, but is not limited to, about 30 minutes to about 2 hours, about 45 minutes to about 1.5 hours, or about 1 hour.

All of the various aspects, embodiments and options described herein can be combined in any and all variations. The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance the following claims and their equivalents.

### EXAMPLES

### Example 1

Table 1 shows a sample formulation for the flexible oral tablets of the present invention.

TABLE 1
Flexible Oral Tablet Formulation

| S# | Excipient | mg/tab | % |
|---|---|---|---|
| 1 | Drug | 10 | 5 |
| 2 | GELATIN, NF (75 BLOOM) | 88 | 44 |
| 3 | SORBITOL, NF | 88 | 44 |
| 4 | ASPARTAME, NF | 6 | 3 |
| 5 | ORANGE FLAVOR (POWDER) | 2 | 1 |
| 6 | STEARIC ACID, NF | 6 | 3 |
| | Total (mg) | 200 | 100 |

1 cm², flat face, round and flexible tablets.

### Example 2

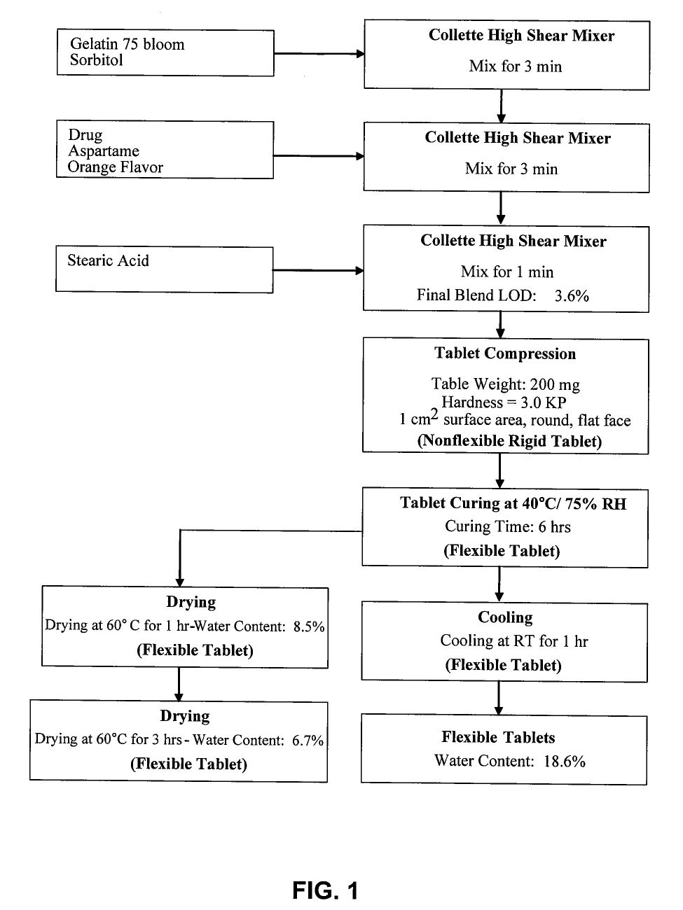

An example of a method of making the flexible oral tablets using the compression method of the present invention follows. First, gelatin (75 Bloom) was mixed with sorbitol in a Collette high shear mixer for 3 minutes. Then a drug, aspartame and orange flavor were mixed, along with the gelatin and sorbitol mixture, in a Collette high shear mixer for 3 minutes. Stearic acid was then added into the mixture and the mixture was mixed in a Collette high shear mixer for 1 minute. The final blend loss on drying (LOD) was 3.6%. The mixture was then compressed into tablets having a tablet weight of 200 mg, a hardness of 3.0 KP, and a 1 cm² round and flat surface area. The tablets were then cured at 40° C. and 75% relative humidity for 6 hours. Some of the cured tablets were dried at 60° C. for 1 hour, after which the water content was 8.5% and the tablets were still flexible (Karl Fischer method). These tablets were then dried again at 60° C. for 3 hour, after which the water content was 6.7% and the tablets were still flexible. Some of the cured tablets were cooled at room temperature for 1 hour, after which the water content was 18.6%. FIG. 1 shows a process flow chart for the method used.

### Example 3

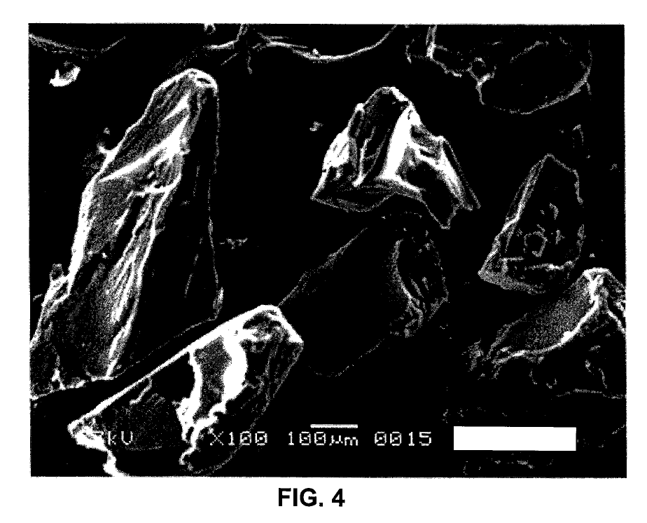

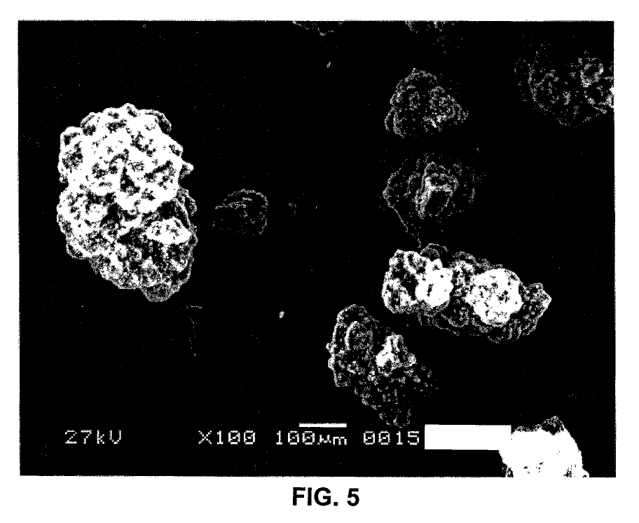

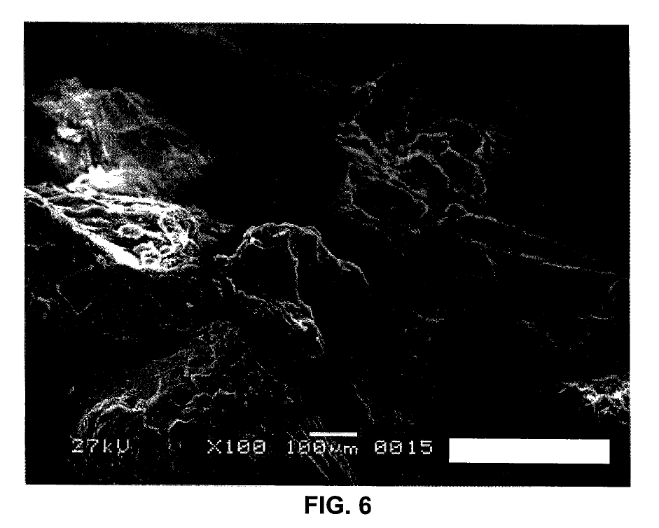

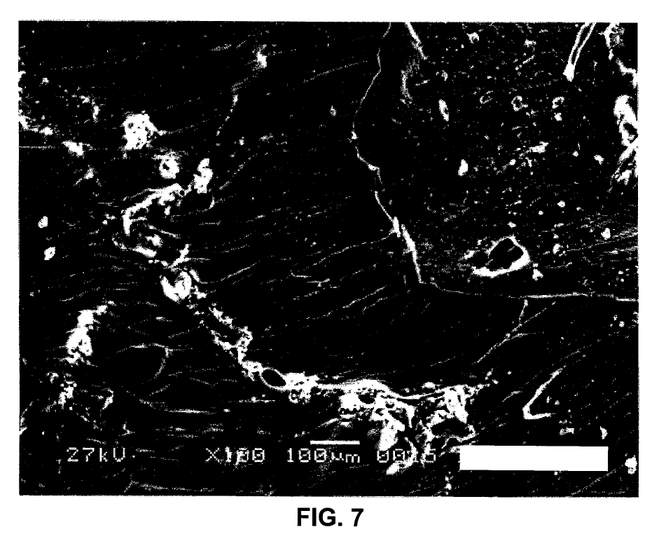

FIGS. 4 and 5 show gelatin and sorbitol granules, respectively, prior to their use in forming the oral tablets. FIG. 6 shows a cross-section of a tablet formed by mixing the gelatin and sorbitol in a 1:1 ratio by weight and compressing the mixture, but without curing the tablet. As can be seen from FIG. 6, distinct granules of gelatin and sorbitol are still present in the mixture. FIG. 7 shows a cross-section of the tablet containing gelatin and sorbitol in a 1:1 ratio by weight after curing. As can be seen from FIG. 7, the distinct granules of gelatin and sorbitol are no longer visible and a substantially homogeneous mixture of the components is achieved.

### Example 4

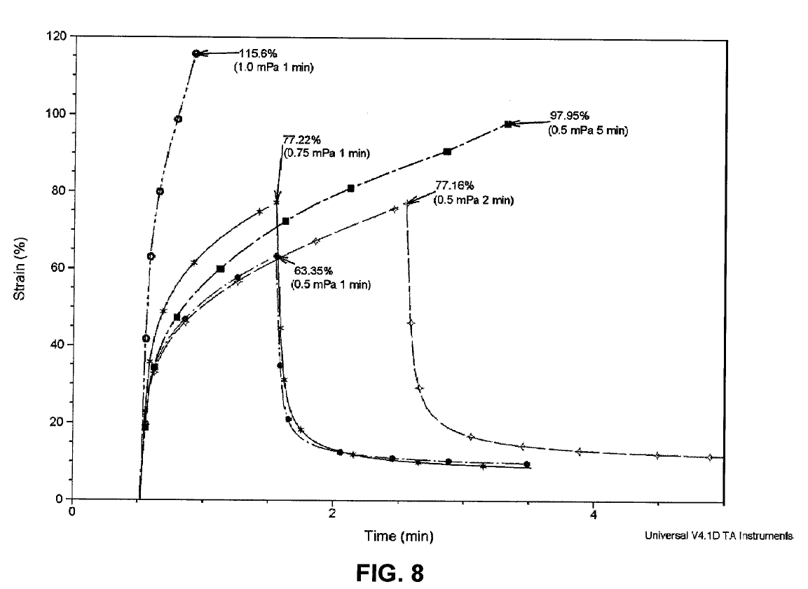

FIG. 8 shows sample strain-time curves obtained from a DMA study using flexible tablets according to the formulation shown in Table 1, but without a drug. The percentage of elongation of a flexible tablet under a stress of 1 millipascal (mPa) for 1 minute was measured to be 115.6%. The percentage of elongation of a flexible tablet under a stress of 0.5 mPa for 5 minutes was measured to be 97.95%. The percentage of elongation of a flexible tablet under a stress of 0.5 mPa for 2 minutes was measured to be 77.16%. The percentage of elongation of a flexible tablet under a stress of 0.75 mPa for 1 minute was measured to be 77.22%. The percentage of elongation of a flexible tablet under a stress of 0.5 mPa for 1 minute was measured to be 63.35%.

### Example 5

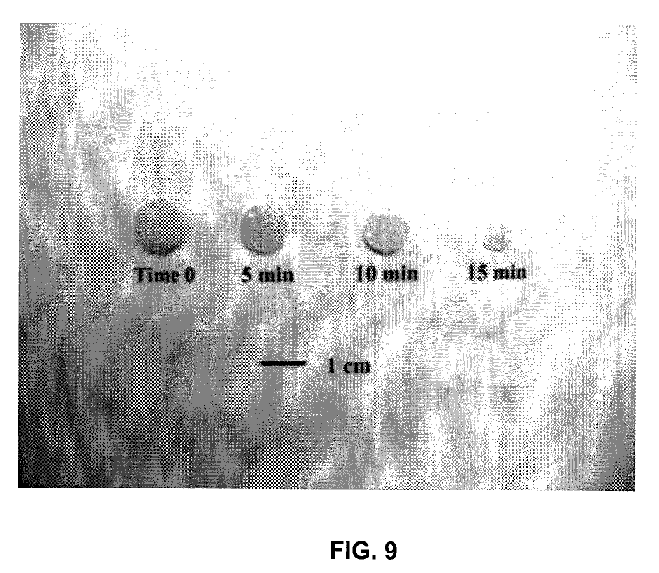

A tablet erosion study of a flexible oral tablet of the present invention was carried out with the USP dissolution apparatus II (paddle method) at 37° C. and 50 rpm in 900 ml water. The flexible oral tablet was made according to the formulation shown in Table 1, but without a drug. The tablet started loosing its integrity after 15 minutes. FIG. 9 shows the erosion of the tablet with time.

### Example 6

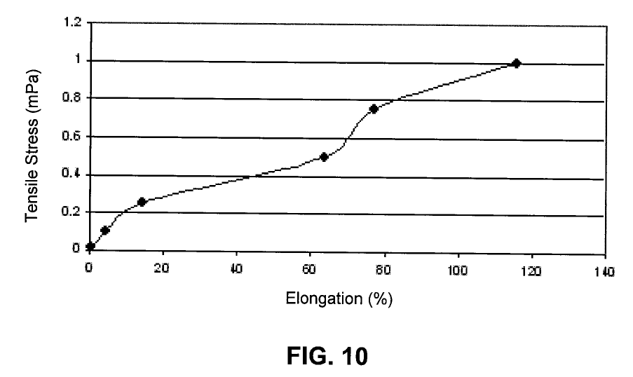

A stress-strain curve of the flexible oral tablets of the present invention was obtained. The flexible oral tablets were made according to the formulation of Table 1, but without a drug. Table 2 below shows the percent strain and stress data obtained. FIG. 10 shows the stress-strain curve obtained.

TABLE 2
% Strain and Stress

| % Strain | Stress (mPa) |
|---|---|
| 0.44 | 0.01 |
| 4.21 | 0.1 |
| 14.31 | 0.25 |
| 63.35 | 0.5 |
| 77.22 | 0.75 |
| 115.6 | 1 |

### Example 7

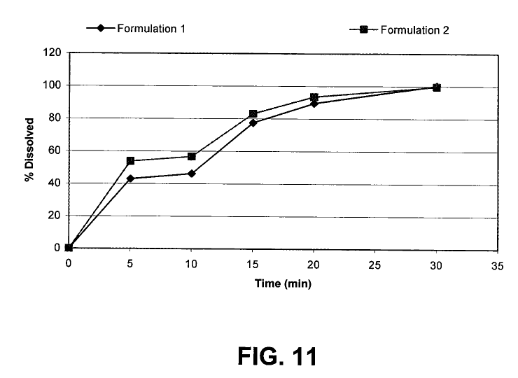

Table 3 below shows two formulations of the flexible oral tablets containing olanzapine as the active agent. Table 4 shows the results of dissolution tests conducted on these olanzapine containing oral tablets using the paddle method at 50 rpm in 500 ml simulated saliva (pH 6.2). The dissolution tests show the amount of olanzapine that was dissolved as a function of time. FIG. 11 is a graph of the dissolution data obtained.

TABLE 3
Olanzapine flexible oral tablet formulations

mg/tab

| Ingredient | Formulation 1 | Formulation 2 |
|---|---|---|
| Olanzapine | 5 | 5 |
| Gelatin (60 bloom, 40 mesh) | 90.5 | 44 |
| Sorbitol (instant powder) | 90.5 | 44 |
| Aspartame | 6 | 3 |
| Orange flavor | 2 | 1 |
| Stearic acid | 6 | 3 |
| Total Weight (mg) | 200 | 100 |

TABLE 4
Dissolution Test

% Olanzapine Dissolved

| Time (min) | Formulation 1 | Formulation 2 |
|---|---|---|
| 0 | 0 | 0 |
| 5 | 43 | 54 |
| 10 | 46 | 57 |
| 15 | 78 | 83 |
| 20 | 89 | 93 |
| 30 | 100 | 100 |

### Example 8

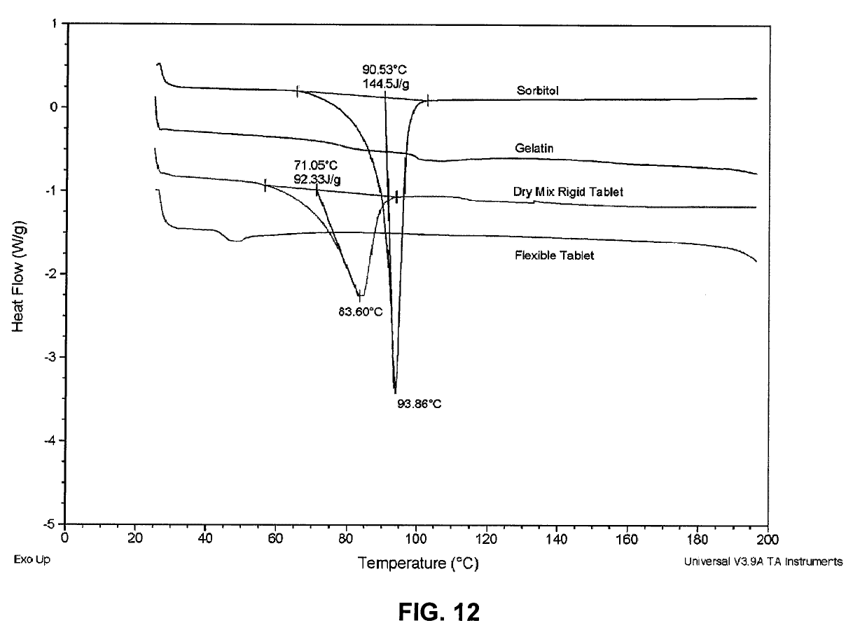

FIG. 12 shows that the polyol or saccharide loses its crystallinity when mixed with gelatin and cured to form an irreversible flexible mass. FIG. 12 shows sample results obtained from a Differential Scanning Calorimetry study. Samples tested included: 1) sorbitol, 2) gelatin, 3) a dry mix of sorbitol and gelatin in a 1:1 ratio by weight that has been compressed into a rigid tablet, before curing, and 4) a flexible tablet containing sorbitol and gelatin in a 1:1 ratio by weight, after curing. The curing was done at 40° C. and 75% relative humidity for 6 hours. Distinct endothermal peaks were observed for the sorbitol sample and for the sample containing a dry mix of sorbitol and gelatin that has been compressed into a rigid tablet, before curing. The endotherms appeared to be due to melting of the sorbitol material. Typically, the melting event of a material is a change from solid to liquid. When sorbitol is melted, it acts as a plasticizer that improves the pliability of a material. The amount of energy needed to break the arrangement of the sorbitol crystalline structure has to be greater than that needed to maintain the crystalline structure. The thermograph of FIG. 12 indicates that at temperatures prior to or below the melting event, the sorbitol was in crystalline form, which existed in the sorbitol sample and in the mixed system of sorbitol and gelatin prior to curing. The shift in melting event of the sorbitol was an indication of the mixed system. However, when the mixed system was cured, the crystalline property of the sorbitol was lost and the mixed material became amorphous.

In the thermograph of the heat flow as a function of temperature, a sorbitol sample had an endothermal peak at 90.53° C., indicating that the sorbitol was absorbing heat as it melted and lost its crystallinity. The gelatin sample, which was not in crystalline form, did not have such a distinct endothermal peak. An endothermal peak was observed at 71.05° C. in the sample containing a dry mix of sorbitol and gelatin before curing, indicating that there was still crystalline sorbitol present, which melted and lost crystallinity after absorbing heat. No distinct endothermal peak was observed for the flexible tablet which was cured, indicating that after curing, there was substantially no crystalline sorbitol remaining. The loss of crystalline sorbitol observed after curing further confirmed that a substantially homogeneous mixture of the gelatin and the polyol or saccharide was achieved after curing, forming an irreversible flexible mass.

### Example 9

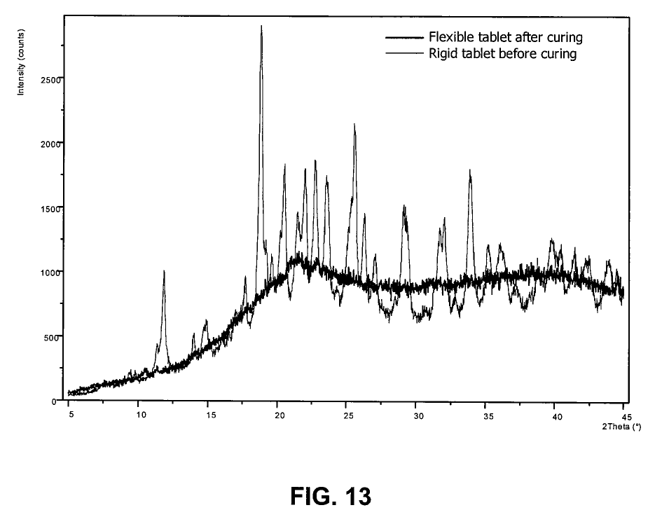

FIG. 13 shows an X-ray diffraction graph of 1) a dry mix of sorbitol and gelatin in a 1:1 ratio by weight that has been compressed into a rigid tablet, before curing, and 2) a flexible tablet containing sorbitol and gelatin in a 1:1 ratio by weight, after curing at 40° C. and 75% relative humidity for 7 hours. In the graph, many peaks were observed for the sample of sorbitol and gelatin before curing. This indicates that there were crystalline sorbitol in the mixture. After curing, no substantial peaks were observed, indicating that crystallinity of the sorbitol in the mixture was lost after curing.

### Example 10

Table 5 shows examples of preservatives that can be added and sample concentrations of the preservatives in the oral tablets of the present invention.

TABLE 5
Preservatives

| | Concentration in % by |
|---|---|
| Preservative | weight of the oral tablet |
| benzalkonium chloride | about 0.2-about 0.5 |
| cetalokonium chloride | about 0.2-about 0.5 |
| benzoates, e.g. sodium benzoates | about 0.2 to about 0.5 |
| benzyl alcohol | about 2 |
| methyl paraben | about 1 |
| propyl paraben | about 0.5 |
| alkaly gallates | about 0.1 |
| ascorbyl palmitate | about 0.1 |
| butylated hydroxy anisole | about 0.02 |
| butylated hydroxy toluene | about 0.02 |
| sodium bi sulphite, sodium meta bi sulphite | about 2.0 |
| hydroxybenzoates, e.g. methyl or propyl | about 1 |
| hydroxybenzoates, and salts thereof | |
| phenyl mercuric salts, e.g. borates or nitrates | about 0.1 |
| sodium hypocholorite | about 1 |
| acetic acid | about 1 |
| potassium meta bi sulphite | about 1 |

### Example 11

Table 6 below shows a formulation for flexible oral tablets containing estradiol as the active agent using the granulation compression and the soaking methods described herein.

TABLE 6
Estradiol flexible oral tablet formulations

mg/tab

| | Formulation 1 | |
|---|---|---|
| | (granulation | Formulation 2 |
| Ingredient | compression tablet) | (soaking tablet) |
| Estradiol | 1 | 0.2 |
| Methylparaben | 0.4 | 0.08 |
| Propylparaben | 1.2 | 0.02 |
| Ethanol | * | * |
| Sorbitol | 94 | 89 |
| Gelatin | 94 | 89 |
| Aspartame | 6 | 7 |
| Strawberry Flavor | 2.5 | 3 |
| Stearic Acid | 2 | 3 |
| MCC (PH 105) | — | 10 |
| Total Weight (mg) | 200 | 200.3 |

* indicates that substance not detected in the final formulation.

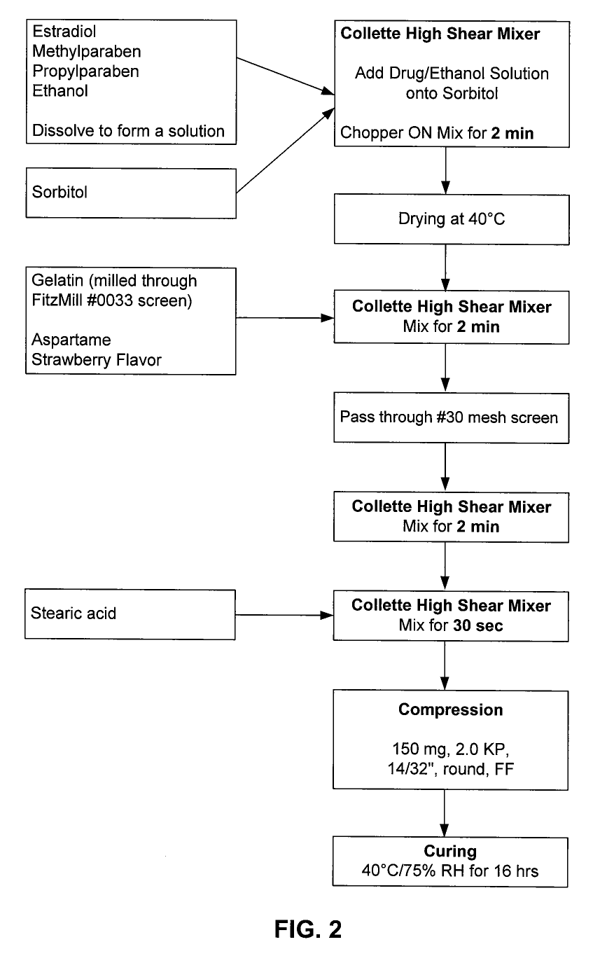

To make a flexible oral tablet by compression, the estradiol, methylparaben, and propylparaben of Formulation 1 were dissolved in ethanol to form a solution. This solution was added with the sorbitol of Formulation 1 to a Collette high shear mixer and mixed for 2 minutes. The resulting granules were then dried at 40° C., then mixed with the gelatin, MCC (PH 105), aspartame, and strawberry flavor of Formulation 1 to form a mixture. This mixture was passed through a #30 mesh screen, then mixed again for 2 minutes in a Collette high shear mixer for 2 additional minutes. Stearic acid was then added to the mixture, followed by mixing for an additional 30 seconds. The resulting mixture was compressed into tablets having a weight of 150 mg, a hardness of 2.0 KP, and a 14/32 inch round flat surface area. The tablets were then cured at 40° C. and 75% relative humidity for 16 hours. FIG. 2 shows a process flow chart for the method used.

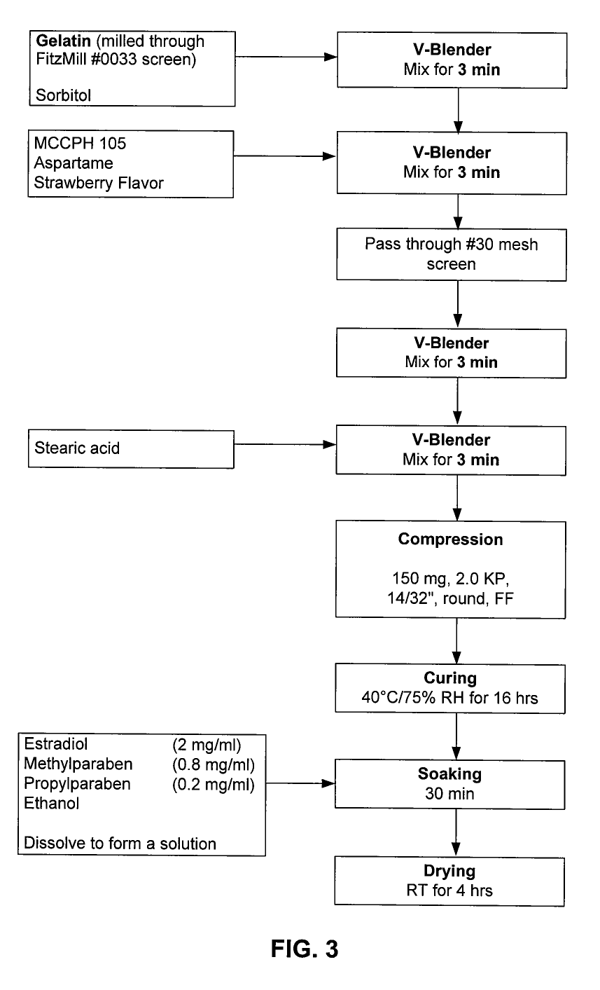

To make a flexible oral tablet using the soaking method, the gelatin and sorbitol of Formulation 2 were mixed in a V-blender for 3 minutes. The MCC (PH 105), aspartame, and strawberry flavor of Formulation 2 were added and mixed for 3 minutes. The resulting mixture was passed through a #30 mesh screen and mixed for an additional 3 minutes. Stearic acid was then added to the mixture, then mixed for 30 seconds. The resulting mixture was compressed into tablets 150 mg tablets having a hardness of 2.0 KP, and a 14/32 inch round flat surface area. The tablets were then cured at 40° C. and 75% relative humidity for 16 hours. The cured tablets were then soaked in an estradiol solution for 3 minutes, the ethanol solution containing estradiol, methylparaben, propylparaben and ethanol. After the tablets had been soaked for 30 minutes, they were dried at room temperature for 4 hours. FIG. 3 shows a process flow chart for the method used.

### Example 12

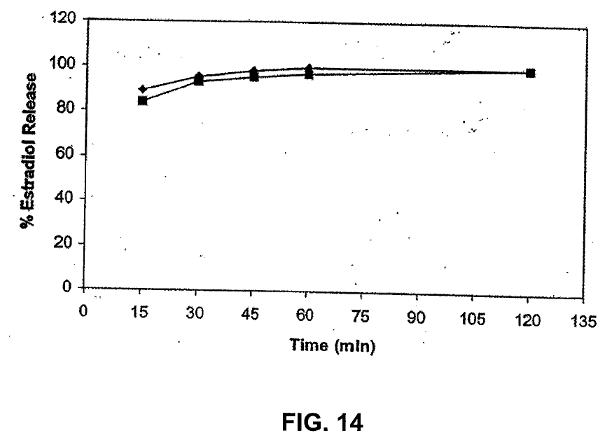

The dissolution profiles of the oral flexible tablets made by the granulation compression method and the soaking method described in Example 11 were determined. The percentage of estradiol released was calculated over time. The dissolution data is provided in FIG. 14. The dissolution profile of the tablet made by the granulation compression method is designated with solid squares. The dissolution profile of the tablet made by the soaking method is designated with solid diamonds.

### Example 13

Table 7 below shows a sample flexible ring formulation of the present invention.

TABLE 7
Flexible Ring Formulation:

| Excipient | g/Ring |
|---|---|
| Gelatin, NF (75 bloom) | 2.82 |
| Sorbitol, NF | 2.82 |
| Colloidal Silicon Dioxide, NF | 0.3 |
| Stearic Acid, NF | 0.06 |
| Total Ring Weight (g) | 6 |

An example of a method of making flexible rings of the present invention follows. Gelatin (75 bloom) was milled through FitzMill fitted with 1522-0033 mesh screen and mixed with sorbitol, followed by colloidal silicon dioxide and stearic acid. The final blend was compressed into rings having a weight of 6 g, an outer diameter of 2.1 inches, an inner diameter of 1.45 inches, and a thickness of 0.27 inches. The rings were cured at 40° C. and 75% relative humidity for 16 hours, and then cooled at room temperature for 30 minutes. The rigid compressed rings were converted into flexible delivery device through the curing process.

It will be appreciated that the various embodiments which have been described above are intended to illustrate the invention and various changes and modifications can be made in the invention method without departing from the spirit or scope thereof.

Having now fully described this invention, it will be understood to those of ordinary skill in the art that the same can be performed within a wide and equivalent range of conditions, compositions, and other parameters without affecting the scope of the invention or any embodiments thereof. All patents, patent applications, and publications cited herein are fully incorporated by reference herein in their entirety.

What is claimed is:

1. A solid dosage form comprising a substantially homogeneous mixture of an active agent, gelatin, and a polyol or saccharide, wherein the solid dosage form is flexible.

2. The solid dosage form of claim 1, wherein the solid dosage form comprises a polyol.

3. The solid dosage form of claim 1, wherein the solid dosage form comprises a saccharide.

4. The solid dosage form of claim 1, wherein the solid dosage form is erosion controlled.

5. The solid dosage form of claim 4, wherein the solid dosage form erodes at a rate of about 0.5 mg/min/cm² to about 10 mg/min/cm².

6. The solid dosage form of claim 1, wherein the active agent is selected from the group consisting of a drug, vitamin, mineral, dietary supplement, and combinations thereof.

7. The solid dosage form of claim 6, wherein the active agent is a drug.

8. The solid dosage form of claim 1, further comprising a sweetener.

9. The solid dosage form of claim 1, further comprising a flavoring agent.

10. The solid dosage form of claim 1, further comprising a lubricant.

11. The solid dosage form of claim 1, further comprising a polymer selected from the group consisting of sodium alginate, carbomer, microcrystalline cellulose, polyvinyl pyrrolidone, polyethylene oxide, glycerin, hydroxypropyl methylcellulose, hydroxypropyl cellulose, hydroxyethyl cellulose, polyvinyl alcohol, sodium carboxymethylcellulose, tragacanth, and combinations thereof.

12. The solid dosage form of claim 1, further comprising an excipient selected from the group consisting of triacetin, triethyl citrate, dibutyl sebacate, castor oil, PEG 400, propylene glycol, and combinations thereof.

13. The solid dosage form of claim 1, wherein the solid dosage form has an elongation of about 10% to about 150%.

14. The solid dosage form of claim 1, wherein the solid dosage form is an oral tablet.

15. The solid dosage form of claim 1, wherein the solid dosage form is a vaginal ring.

16. The solid dosage form of claim 1, wherein the solid dosage form further comprises a coating.

17. The solid dosage form of claim 16, wherein the coating comprises hydroxypropyl methylcellulose, hydroxypropyl cellulose, or a combination thereof.

18. A method of making a flexible solid dosage form, the method comprising:
(a) mixing an active agent, gelatin, and a polyol or saccharide to form a substantially homogenous mixture;
(b) compressing the substantially homogeneous mixture into a solid dosage form; and
(c) curing the solid dosage form.

19. The method of claim 18, comprising mixing a polyol to form a substantially homogeneous mixture.

20. The method of claim 18, comprising mixing a saccharide to form a substantially homogeneous mixture.

21. The method of claim 18, further comprising mixing a sweetener into the mixture prior to compressing the mixture.

22. The method of claim 18, further comprising mixing a flavoring agent into the mixture prior to compressing the mixture.

23. The method of claim 18, further comprising mixing a lubricant into the mixture prior to compressing the mixture.

24. The method of claim 18, further comprising mixing a polymer selected from the group consisting of sodium alginate, carbomer, microcrystalline cellulose, polyvinyl pyrrolidone, polyethylene oxide, glycerin, hydroxypropyl methylcellulose, hydroxypropyl cellulose, hydroxyethyl cellulose, polyvinyl alcohol, sodium carboxymethylcellulose, tragacanth, and combinations thereof, into the mixture prior to compressing the mixture.

25. The method of claim 18, further comprising mixing an excipient selected from the group consisting of triacetin, triethyl citrate, dibutyl sebacate, castor oil, PEG 400, propylene glycol, and combinations thereof, into the mixture prior to compressing the mixture.

26. The method of claim 18, wherein the mixing is carried out using a high shear mixer.

27. The method of claim 18, wherein the curing is carried out at about 30° C. to about 60° C.

28. The method of claim 18, further comprising drying the solid dosage form.

29. The method of claim 18, further comprising cooling the solid dosage form.

30. A method of making a flexible solid dosage form, the method comprising:
(a) dissolving an active agent in a solvent to form a solution;
(b) mixing the solution with a polyol or saccharide to form granules;
(c) drying and mixing the granules with gelatin;
(d) compressing the mixture into a solid dosage form; and
(e) curing the solid dosage form.

31. A method of making a flexible solid dosage form, the method comprising:
(a) mixing a gelatin, and a polyol or saccharide to form a substantially homogenous mixture;
(b) compressing the substantially homogeneous mixture into a solid dosage form;
(c) curing the solid dosage form; and
(d) soaking the cured solid dosage form in a solution comprising an active agent.

32. The solid dosage form of claim 1, further comprising a preservative.

33. A solid dosage form made by the method of claim 18.

34. A solid dosage form made by the method of claim 30.

35. A solid dosage form made by the method of claim 31.
